(19) United States
(12) Patent Application Publication
(10) Pub. No.: US 2016/0101000 A1
(43) Pub. Date: Apr. 14, 2016

(54) Disposable Foil Lined Personal Bag

(71) Applicant: Tyra Michelle Andrews Lopez, Stamford, CT (US)

(72) Inventor: Tyra Michelle Andrews Lopez, Stamford, CT (US)

(21) Appl. No.: 14/509,886

(22) Filed: Oct. 8, 2014

(51) Int. Cl. A61F 13/551; B65D 30/08; B65D 33/25; B65F 1/00; B65D 33/18

(52) U.S. Cl. CPC A61F 13/551; B65F 1/0006; B65D 33/18; B65D 33/2508; B65D 31/04; A61F 2013/8408

(57) ABSTRACT

A disposable foil lined top closure sealable hygiene bag consisting of an outer and inner layer with an interior space; to allow for the disposal of personal hygiene products, personal care, and consumer products. The outer layer consists of a paper or plastic based material. The outer layer can vary in degrees of transparency, opacity, and color. The inner layer is a foil based material attached and lined to the outer layer and exposed in the interior of the sealable bag.

### CROSS-REFERENCE TO RELATED APPLICATIONS

### Patent No., Filing Date

Patent Application—US 2006/0045391 A1, Mar. 2, 2006
U.S. Pat. No. 6,702,116 B2, Mar. 9, 2004
U.S. Pat. No. 6,059,100, May 9, 2000
U.S. Pat. No. 4,349,104, Sep. 14, 1982
Patent Application—US 2007/0055213 A1, Mar. 8, 2007
U.S. Pat. No. 5,950,818, Sep. 14, 1999
U.S. Pat. No. 8,282,280 B2, Oct. 9, 2012
U.S. Pat. No. D494,852 S, Aug. 24, 2004
Patent Application—US 2011/0229059 A1, Sep. 22, 2011
Patent Application—US 2010/0094238 A1, Apr. 15 2010
WO 2007/050928, May 3, 2007

### STATEMENT REGARDING FEDERALLY SPONSORED RESEARCH OR DEVELOPMENT (IF APPLICABLE)

Not Applicable

### REFERENCE TO SEQUENCE LISTING, A TABLE, OR A COMPUTER PROGRAM LISTING COMPACT DISC APPENDIX (IF APPLICABLE)

Not Applicable

### BACKGROUND OF THE INVENTION

Solving the problem of disposing of personal hygiene products such as tampons and panty liners in a neat discreet and sanitary method, every month is a challenge for many menstruating women from the ages on average of 12 to 49 years old. This challenge also presents itself to many men and women of varying ages every time they use other personal products such as condoms, suppositories, and incontinence products.

Historically, individuals have had to wrap the personal product in toilet paper in order to attempt to disguise the used product with no means to mask the odor or type of object being thrown away. Most times the personal product soaks through or breaks the toilet paper and does not conceal the personal product and often times reveals biological matter to other individuals. This exposure of the used personal products causes embarrassment to the individual and intrusion of their personal space and privacy. The exposure can also be a health risk to other individuals upon discovery of the used personal product.

### BRIEF SUMMARY OF THE INVENTION (ADVANTAGES, PROBLEMS SOLVED)

The disposable foil lined sealable personal bag is a new type of disposable bag that allows all types of personal products to be disposed of in a shameless hygienic manner. This leak-proof, odor concealing product provides the discreet containment and disposal of all types of personal products including but not limited to; tampons, sanitary pads, panty liners, condoms, suppositories, personal health test strips, and incontinence products.

The disposable sealable personal bag is foil lined and consists of multiple material layers with a top closure. The benefits of the foil lined bags include but are not limited to; aiding in clean hygienic disposal, increased durability of the bag, odor suppression, and the containment and masking of personal products.

The sealable bag varies in size, shape, degrees of transparency, opacity, and color. The flexible variations of size, color, layering technology allows for the increased number and types of personal products that may be disposed of in a discreet manner tailored to individual tastes and needs.

Upon usage of personal products, an individual would place the personal product inside of the interior of the disposable foil-lined bag, seal the bag, and discard the contained matter.

The disposable foil lined bag fulfills the need for a sanitary means of concealing and discreetly disposing of personal products.

The appealing features of the disposable foil lined bags include but are not limited to its convenience, ease of use, portability, effectiveness, light weight, compact size, and practicality. The disposable foil lined bags provide individuals a method to contain and dispense their personal products in a quick, neat, discreet, and sanitary way in a private or public bathroom. The disposable foil lined bags supply an inconspicuous containment method with leak prevention, and an air trapping sealable system to assist in reducing germs and offensive odors for the masking and discarding of personal products. The disposable foil lined bags are lightweight and compact to be easily stored and transported when needed making clean and sanitary bathrooms available for private and public use.

### BRIEF DESCRIPTION OF THE SEVERAL VIEWS OF THE DRAWING

FIG. 1—Disposable Foil-Lined Personal Bag—Front/Back View—Outer Layer

FIG. 2—Disposable Foil-Lined Personal Bag—Front/Back View—Inner Layer

FIG. 3—Disposable Foil-Lined Personal Bag Layers

FIG. 4—Disposable Foil-Lined Personal Bag—Side View

FIG. 5—Disposable Foil-Lined Personal Bag—Top View

### DETAILED DESCRIPTION OF THE INVENTION

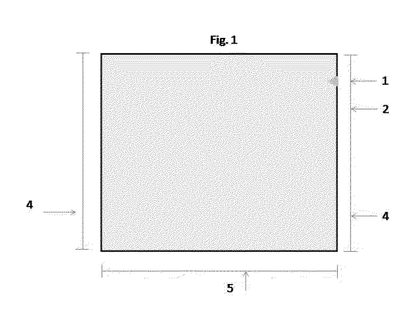

FIG. 1—Disposable Foil-Lined Personal Bag—Front/Back View

The front and back view minor each other and details the sealable foil lined bags' open, top closure, sealed side and sealed bottom features.
1—Open—The sealable foil lined bag can consist of an open wedge portion that is removed from the sides of the bag to aid in bag manipulation. The sealable foil lined bag can also consist without an open wedge portion removed from the sides of the bag.
2—Top Closure—The sealable foil lined bag closure is placed at the top of the bag. The closure mechanisms to seal the bag include but are not limited to zip-lined, lippers, adhesive, zippers or other mechanisms for sealing the bag. The sealing mechanism is within the interior of the bag. The top closure opens and closes by the user of the bag to receive the product and seal the bag.
4—Sealed Side—Each length side of the sealable foil lined bag is sealed by heat or other manufacturing mechanism during the manufacturing process. The sealed length side cannot be open or closed by the user of the bag and acts part of the containment mechanism of the bag.
5—Sealed Bottom—The bottom of the sealable foil lined bag is sealed by heat or other manufacturing mechanism during the manufacturing process. The sealed bottom side cannot be open or closed by the user of the bag and acts part of the containment mechanism of the bag.

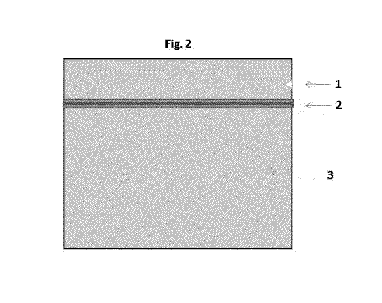

FIG. 2—Disposable Foil-Lined Personal Bag—Inner View

The inner view mirror details the sealable foil lined bags' open, top closure, and inner foil lining features.
1—Open—The sealable foil lined bag can consist of an open wedge portion that is removed from the sides of the bag to aid in bag manipulation. The sealable foil lined bag can also consist without an open wedge portion removed from the sides of the bag.
2—Top Closure—The sealable foil lined bag closure is placed at the top of the bag. The closure mechanisms to seal the bag include but are not limited to zip-lined, lippers, adhesive, zippers or other mechanisms for sealing the bag. The sealing mechanism is within the interior of the bag.
3—Inner Foil Lining—The inner layer of the sealable bag consists of a foil based material. The inner layer of the sealable bag is fused together to the outer layer as an inner lining. The foil based inner layer of the sealable bag is a method of receiving the materials, containing odors and masking the receivables.

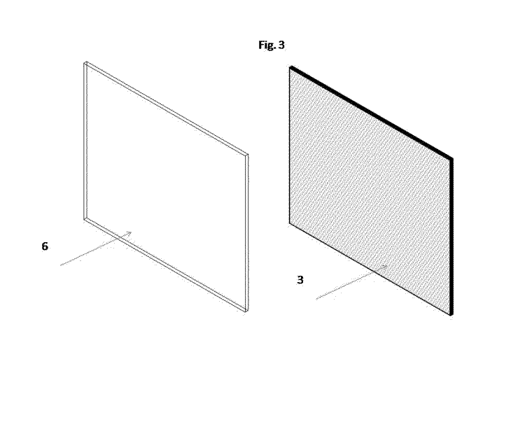

FIG. 3—Disposable Foil-Lined Personal Bag—Layers View

The layer view details the sealable foil lined bags' inner foil lining, and outer layer features.
3—Inner Foil Lining—The inner layer of the sealable bag consists of a foil based material. The inner layer of the sealable bag is fused together to the outer layer as an inner lining. The foil based inner layer of the sealable bag is a method of receiving the materials, containing odors and masking the receivables.
6—Outer Layer—The outer layer of the sealable bag consists of a plastic or paper based material or other combination of materials.

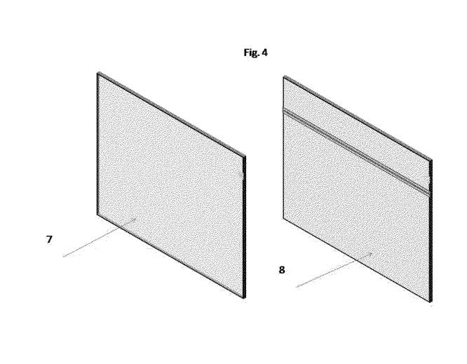

FIG. 4—Disposable Foil-Lined Personal Bag—Side View

The side view details the sealable foil lined bags' outside and inside view.
7—Outside View—The outside view of the sealable foil lined bag consists of the outer layer of material.
8—Inside View—The inside view of the sealable foil lined bag consists of the inner foil layer and the closure mechanism.

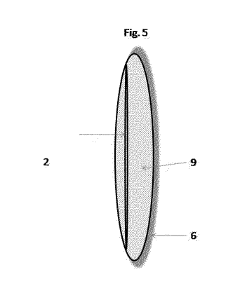

FIG. 5—Disposable Foil-Lined Personal Bag—Top View

The layer view details the sealable foil lined bags' top closure, outer layer and inner foil container features.
2—Top Closure—The sealable foil lined bag closure is placed at the top of the bag. The closure mechanisms to seal the bag include but are not limited to zip-lined, lippers, adhesive, zippers or other mechanisms for sealing the bag. The sealing mechanism is within the interior of the bag.
6—Outer Layer—The outer layer of the sealable bag consists of a plastic or paper based material or other combination of materials.
9—Inner Foil Container—The inner portion of the sealable bag consists of a foil based material. The foil based inner container of the sealable bag is a method of receiving the materials, controlling odors and masking the receivables.

1) A storage and disposal system with a foil lined disposal bag for personal hygiene products, personal care, and consumer products comprising of;
A. Sealable bag varies in size, thickness, and depth with each side surface composed of a minimum of two layers, an outer and inner layer fused together.
B. Sealable bag consists of a top closure.
C. Sealable bag consists with an open wedge portion removed from the sides of the bag to aid in bag manipulation and without an open wedge portion removed from the sides of the bag.
D. The sealable opening mechanisms include but are not limited to zip-lined, lippers, adhesive, zippers or other mechanisms for sealing the bag. The sealing mechanism is within the interior of the bag.
E. Three sides of the sealable bag are sealed by heat or other manufacturing mechanism.
F. The sealable bag may be impregnated with scents, odor-eliminating, and/or substance degrading mechanisms.
G. The outer layer of the sealable bag consists of a plastic or paper based material or other combination of materials.
H. The outer layer of the sealable bag varies in degrees of transparency, opacity, and color.
I. The inner layer of the sealable bag consists of a foil based material.
J. The inner layer of the sealable bag is fused together to the outer layer as an inner lining.
K. The foil based inner layer of the sealable bag is a method of receiving and containing the materials, controlling odors and masking the receivables.
